(19) United States
(12) Patent Application Publication
(10) Pub. No.: US 2019/0330319 A1
(43) Pub. Date: Oct. 31, 2019

(54) STABLE AQUEOUS ANTI-C5 ANTIBODY COMPOSITION

(71) Applicant: SAMSUNG BIOEPIS CO., LTD, Incheon (KR)

(72) Inventors: Jaemin LEE, Incheon (KR); Yuna SHON, Incheon (KR); Tae-Soo LEE, Incheon (KR); Brian Ho Sung MIN, Incheon (KR); YongKook KIM, Incheon (KR); Soojeong PARK, Incheon (KR); Sungjae PARK, Incheon (KR)

(73) Assignee: SAMSUNG BIOEPIS CO., LTD, Incheon (KR)

(21) Appl. No.: 16/470,047

(22) PCT Filed: Nov. 22, 2017
(86) PCT No.: PCT/IB2017/057348

§ 371 Date: Jun. 14, 2019

Related U.S. Application Data
(60) Provisional application No. 62/435,476, filed on Dec. 16, 2016.

(51) Int. Cl. C07K 16/18; A61K 31/7004; A61K 31/198

(52) U.S. Cl. CPC C07K 16/18; C07K 2317/24; A61K 31/198; A61K 31/7004

(57) ABSTRACT

A stable aqueous composition comprising (a) about 10 to about 100 mg/ml of an anti-C5 antibody, (b) a surfactant, (c) a stabilizer, and (d) a buffer having a pH of about 5.0 to about 7.8, wherein the stabilizer is trehalose, sucrose, sorbitol, arginine, or a combination thereof is provided. In a particular embodiment, the stable aqueous composition consists essentially of (a) about 10 or about 50 mg/ml of an anti-C5 antibody, (b) about 0.01 to about 0.1% (w/v) of a surfactant, (c) about 1 to about 20 mM buffer having a pH of 5.5 to 7.5, and (d) a stabilizer selected from the group consisting of trehalose, sucrose, sorbitol, arginine, or a combination thereof. A method of treating a disorder in which C5 activity is detrimental in a subject comprising administering the stable aqueous composition to the subject also is provided.

### SEQUENCE LISTING

Incorporated by reference in its entirety herein is a nucleotide/amino acid sequence listing submitted concurrently herewith.

### BACKGROUND OF THE INVENTION

As part of the innate immune system, the complement system recognizes a wide range of non-self structures present on pathogens or altered self cells. Its activation elicits proteolytic cascades which result in the cleavage of the C5 protein into two fragments: C5a and C5b. The small anaphylatoxin C5a induces a variety of biological responses upon binding to the 7TM receptors C5aR and the C5L2, while the large C5b fragment nucleates formation of the membrane attack complex capable of killing susceptible pathogens by the formation of a pore structure in association with complement components C6, C7, C8, and C9 (see Laursen et al., Curr. Mol. Med., 12(8): 1083-97 (2012)).

Eculizumab (SOLIRIS™, Alexion) is a humanized IgG2/4 kappa monoclonal antibody that binds to the human complement protein C5 with high affinity, thereby inhibiting its cleavage to C5a and C5b and preventing generation of the terminal complement complex C5b-9. The eculizumab heavy and light chain sequences are described in U.S. Patent Application Publication 2009/0220508.

Eculizumab inhibits terminal complement mediated intravascular hemolysis in patients with paroxysmal nocturnal hemoglobinuria (PNH). PNH is a debilitating and life-threatening, ultra-rare genetic blood disorder defined by chronic uncontrolled complement activation leading to the destruction of red blood cells (hemolysis). The chronic hemolysis in patients with PNH may be associated with life-threatening thromboses, recurrent pain, kidney disease, disabling fatigue, impaired quality of life, severe anemia, pulmonary hypertension, shortness of breath and intermittent episodes of dark-colored urine (hemoglobinuria). Eculizumab (SOLIRIS™, Alexion) has been granted orphan drug designation for the treatment of PNH in the United States, Europe, Japan and several other territories.

Eculizumab (SOLIRIS™, Alexion) also is approved for the treatment of pediatric and adult patients with atypical hemolytic uremic syndrome (aHUS) in the United States, Europe and Japan. aHUS is a severe and life-threatening genetic ultra-rare disease characterized by chronic uncontrolled complement activation and thrombotic microangiopathy (TMA), the formation of blood clots in small blood vessels throughout the body, causing a reduction in platelet count (thrombocytopenia) and life-threatening damage to the kidney, brain, heart and other vital organs. Eculizumab (SOLIRIS™, Alexion) has been granted orphan drug designation for the treatment of aHUS in the United States and Europe.

There is a desire for stable aqueous compositions of an anti-C5 antibody (e.g., eculizumab) that allow long term storage without substantial loss in efficacy.

### BRIEF SUMMARY OF THE INVENTION

The invention provides a stable aqueous composition comprising consisting essentially of, or consisting of (a) about 10 to about 100 mg/ml of an anti-C5 antibody, (b) a surfactant, (c) a stabilizer, and (d) a buffer having a pH of about 5.0 to about 7.8, wherein the stabilizer is trehalose, sucrose, sorbitol, arginine, or a combination thereof.

The invention also provides a stable aqueous composition consisting essentially of (a) about 10 mg/ml of an anti-C5 antibody, (b) about 0.01 to about 0.1% (w/v) of a surfactant, (c) about 1 to about 20 mM buffer having a pH of 5.5 to 7.5, and (d) a stabilizer selected from the group consisting of trehalose, sucrose, sorbitol, arginine, or a combination thereof. Such a composition is suitable for administration (e.g., intravenous administration) to a subject. As such, the invention provides a method of treating a disorder in which C5 activity is detrimental in a subject, comprising administering the stable aqueous composition to the subject, thereby treating the disorder in the subject.

The invention provides a stable aqueous composition consisting essentially of (a) about 50 mg/ml of an anti-C5 antibody, (b) about 0.01 to about 0.1% (w/v) of a surfactant, (c) about 1 to about 20 mM buffer having a pH of 5.5 to 7.5, and (d) a stabilizer selected from the group consisting of trehalose, sucrose, sorbitol, arginine, or a combination thereof. Stable aqueous compositions containing high concentrations (e.g., 50 mg/ml) of the anti-C5 antibody can be useful for storage.

### DETAILED DESCRIPTION OF THE INVENTION

The invention provides a stable aqueous composition comprising an anti-C5 antibody, wherein the composition has increased stability relative to conventional anti-C5 antibody formulations even at higher anti-C5 concentrations relative to conventional anti-C5 antibody formulations.

In particular, the invention provides a stable aqueous composition comprising, consisting essentially of, or consisting of (a) about 10 to about 100 mg/ml of an anti-C5 antibody, (b) a surfactant, (c) a stabilizer, and (d) a buffer having a pH of about 5.0 to about 7.8, wherein the stabilizer is trehalose, sucrose, sorbitol, arginine, or a combination thereof.

The anti-C5 antibody can be any suitable antibody or fragment thereof, which can be prepared by any conventional method. Antibodies are found in multiple forms, e.g., IgA, IgG, and IgM, and can be engineered in numerous ways (e.g., as single-chain antibodies, Fab Fab′, (Fab′)₂, Fv, and scFv fragments, diabodies, bispecific or multispecific antibodies). Antibodies can be humanized, chimerized, deimmunized, or fully human Numerous publications set forth the many types of antibodies and the methods of engineering such antibodies (see, e.g., U.S. Pat. Nos. 6,355,245; 6,180,370; 5,693,762; 6,407,213, 6,548,640; 5,565,332; 5,225,539; 6,103,889; and 5,260,203). Preferably, the anti-C5 antibody is a humanized antibody.

In one embodiment, the anti-C5 antibody has a molecular weight of about 145-150 (e.g., about 145, about 146, about 147, about 148, about 149, about 150, or a range between any of these values) kDa. Preferably, the anti-C5 antibody has a molecular weight of about 148 kDa.

In another embodiment, the anti-C5 antibody comprises a light chain variable region comprising complementary determining region (CDR) 1 domain comprising the amino acid sequence of SEQ ID NO: 5; a CDR2 domain comprising the amino acid sequence of SEQ ID NO: 6; and a CDR3 domain comprising the amino acid sequence of SEQ ID NO: 7; and a heavy chain variable region comprising CDR1 domain comprising the amino acid sequence of SEQ ID NO: 8; a CDR2 domain comprising the amino acid sequence of SEQ ID NO: 9; and a CDR3 domain comprising the amino acid sequence of SEQ ID NO: 10. The light chain variable region of the anti-C5 antibody can comprise the amino acid sequence of SEQ ID NO: 3, and a heavy chain variable region can comprise the amino acid sequence of SEQ ID NO: 4.

In a particular embodiment, the anti-C5 antibody is eculizumab and contains the light and heavy chains of SEQ ID NOs: 1 and 2, respectively.

SEQUENCE LISTING

SEQ ID NO: 1 (light chain wherein the variable
region is shown in bold text and the CDRs are
underlined)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 2 (heavy chain wherein the variable
region is shown in bold text and the CDRs are
underlined))
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 3 (light chain variable region wherein
the CDRs are underlined)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRT
SEQ ID NO: 4 (heavy chain variable region wherein
the CDRs are underlined)
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSA
SEQ ID NO: 5 (CDRL1)
GASENIYGALN
SEQ ID NO: 6 (CDRL2)
GATNLAD
SEQ ID NO: 7 (CDRL3)
QNVLNTPLT
SEQ ID NO: 8 (CDRH1)
NYWIQ
SEQ ID NO: 9 (CDRH2)
EILPGSGSTEYTENFKD
SEQ ID NO: 10 (CDRH3)
YFFGSSPNWYFDV

The composition can comprise any suitable amount of the anti-C5 antibody, such as about 10 to about 100 (e.g., about 10, about 20, about 30, about 40, about 50, about 60, about 70, about 80, about 90, about 100, or a range between any of these values) mg/ml of the anti-C5 antibody. For example, the composition can comprise about 30 to about 100 mg/ml of the anti-C5 antibody, about 40 to about 80 mg/ml of the anti-C5 antibody, about 40 to about 60 mg/ml of the anti-C5 antibody, or about 50 mg/ml of the anti-C5 antibody.

The anti-C5 antibody (e.g., eculizumab) can be produced in a known manner by recombinant DNA technology in a mammalian cell such as a mouse myeloma NS0 cell, CHO (e.g., CHO K1 and CHO DUKK) cell, DG44 cell, HEK cell, HEK 293 cell, PER.C6 cell, HeLa cell, and MDCK cell. For example, an expression plasmid containing nucleic acid sequences encoding the light chain and heavy chain (e.g., SEQ ID NOs: 1 and 2, respectively) can be transfected into NS0 cells obtained from a cell bank. The cells are adapted to suspension culture and, following cloning and subcloning steps, one clone is selected as the lead cell line. The antibody can be manufactured in a large scale (e.g., 500 L) production bioreactor.

The surfactant can be any suitable surfactant, such as polysorbate (e.g., polysorbate 20 or polysorbate 80), other fatty acid esters of sorbitan polyethoxylates, and poloxamer 188. In a particular embodiment, the surfactant is polysorbate 80. Any suitable amount of surfactant can be included in the composition. For example, about 0.01% to about 0.1% (e.g., about 0.01%, about 0.015%, about 0.02%, about 0.025%, about 0.030%, about 0.035%, about 0.04%, about 0.045%, about 0.05%, about 0.055%, about 0.06%, about 0.065%, about 0.07%, about 0.075%, about 0.08%, about 0.085%, about 0.09%, about 0.095%, about 0.1%, or a range between any of these values) (w/v) or about 0.01 to about 0.04% (e.g., about 0.01%, about 0.015%, about 0.02%, about 0.025%, about 0.030%, about 0.035%, about 0.04%, or a range between any of these values) (w/v) surfactant (e.g., polysorbate 80) can be included in the composition. In a particular embodiment, the surfactant is about 0.022% (w/v) polysorbate 80.

The buffer can be any suitable buffer having a pH of about 5.0 to about 7.8 and preferably a pH of about 5.5 to about 7.5 (e.g., a pH of 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, or a range between any of these values). In one embodiment, the buffer has a pH of 7.0.

Suitable buffers can comprise phosphate, histidine, or a combination thereof. Preferably, the buffer does not comprise glycine.

In one embodiment, the buffer comprises phosphate (e.g., sodium phosphate). Any suitable phosphate (e.g., sodium phosphate) can be used. The buffer can comprise about 1 mM to about 30 mM (e.g., about 1 mM, about 5 mM, about 10 mM, about 15 mM, about 20 mM, about 25 mM, about 30 mM, or a range between any of these values, such as about 1 mM to about 20 mM) or about 5 mM to about 15 mM (e.g., about 5 mM, about 10 mM, about 15 mM, or a range between any of these values) phosphate (e.g., sodium phosphate). In a particular embodiment, the buffer comprises 10 mM phosphate (e.g., sodium phosphate)

In another embodiment, the buffer comprises histidine. The buffer can comprise about 1 mM to about 30 mM (e.g., about 1 mM, about 5 mM, about 10 mM, about 15 mM, about 20 mM, about 25 mM, about 30 mM, or a range between any of these values, such as about 1 mM to about 20 mM) or about 5 mM to about 15 mM (e.g., about 5 mM, about 10 mM, about 15 mM, or a range between any of these values) histidine. In a particular embodiment, the buffer comprises 10 mM histidine.

The stabilizer is selected from the group consisting of trehalose, sucrose, sorbitol, arginine, or a combination thereof. In one embodiment, the stabilizer is arginine. In another embodiment, the stabilizer is trehalose. Any suitable amount of the stabilizer can be included in the composition.

For example, when the stabilizer is trehalose, the composition can comprise about 1% to about 20% (e.g., about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, or a range between any of these values) (w/v) or about 7.6% to about 11.4% (e.g., about 7.6%, about 8%, about 8.5%, about 9%, about 9.5%, about 10%, about 10.5%, about 11%, about 11.4%, or a range between any of these values) (w/v) trehalose. In one embodiment, the composition comprises about 9.5% (w/v) trehalose.

When the stabilizer is sorbitol, the composition can comprise about 1% to about 20% (e.g., about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, or a range between any of these values) (w/v) or about 4% to about 6% (e.g., about 4%, about 4.5%, about 5%, about 5.5%, about 6%, or a range between any of these values) (w/v) sorbitol. In one embodiment, the composition comprises about 5% (w/v) sorbitol.

When the stabilizer is sucrose, the composition can comprise about 1% to about 20% (e.g., about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, or a range between any of these values) (w/v) or about 6% to about 11% (e.g., about 6%, about 6.5%, about 7%, about 7.5%, about 8%, about 8.5%, about 9%, about 9.5%, about 10%, about 10.5%, about 11% or a range between any of these values) (w/v) sucrose. In one embodiment, the composition comprises about 8.5% (w/v) sucrose.

When the stabilizer is arginine, the composition can comprise about 50 mM to about 300 mM (e.g., about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, or a range between any of these values) or about 120 mM to about 180 mM (e.g., about 120 mM, about 130 mM, about 140 mM, about 150 mM, about 160 mM, about 170 mM, about 180 mM, or a range between any of these values) arginine. In one embodiment, the composition comprises about 150 mM arginine.

When the stable aqueous composition contains 10 mg/ml of an anti-C5 antibody with pH of about 5.5 (e.g., 10 mg/ml eculizumab formulated with the stabilizer trehalose, sucrose, sorbitol, arginine, or a combination thereof), the composition also has increased stability as compared to an anti-C5 antibody in the same amount (10 mg/me formulated in 10 mM histidine buffer, 150 mM sodium chloride, 0.022% polysorbate 80, pH 5.5. The stability can be measured by any suitable means, for example, the increase in the percent of high molecular weight (HMW) aggregate products as determined by size exclusion chromatography (SEC) (1) after four or eight weeks of thermal stress at 40° C. or (2) after five cycles of freeze/thaw (−70° C./RT).

Accordingly, the invention also provides a method of analyzing HMW aggregate products by performing SEC at 0, 1, 2, 4 and/or 8 weeks of thermal stress at 40° C. or at 0, 1, 3, and/or 5 cycles of freeze/thaw (−70° C./RT) and determining the change in HMW aggregate products.

In comparison with a composition containing 10 mg/ml of the antibody formulated in 10 mM histidine buffer, 150 mM sodium chloride, 0.022% polysorbate 80, pH 5.5, the stable aqueous composition comprising 10 mg/ml of anti-C5 antibody (e.g., 10 mg/ml eculizumab formulated with the stabilizer trehalose, sucrose, sorbitol, arginine, or a combination thereof) with pH of about 5.5 has less of an increase in the percent of HMW aggregate products as determined by SEC (1) after four or eight weeks of thermal stress at 40° C. or (2) after five cycles of freeze/thaw (−70° C./RT). For example, the increase in the percent HMW aggregate products in the stable aqueous composition comprising 10 mg/ml of anti-C5 antibody (e.g., 10 mg/ml eculizumab formulated with the stabilizer trehalose, sucrose, sorbitol, arginine, or a combination thereof) can be reduced by about 10% or more (e.g., about 10% or more, about 12% or more, about 13% or more, about 15% or more, about 20% or more, about 25% or more, about 30% or more, about 35% or more, about 39% or more, about 40% or more, about 45% or more, about 50% or more, about 55% or more, about 58% or more, about 59% or more, about 60% or more, about 65% or more, about 70% or more, about 73% or more, about 74% or more, about 75% or more, about 80% or more, about 85% or more, about 90% or more, about 95% or more, about 100% or a range between any of these values) or by about 10% to about 80% (e.g., about 10%, about 12%, about 13%, about 15%, about 20%, about 25%, about 30%, about 35%, about 39%, about 40%, about 45%, about 50%, about 55%, about 58%, about 59%, about 60%, about 65%, about 70%, about 73%, about 74%, about 75%, about 80%, about 85%, about 90%, about 95%, about 100% or a range between any of these values) as compared to a composition containing 10 mg/ml of the antibody formulated in 10 mM histidine buffer, 150 mM sodium chloride, 0.022% polysorbate 80, pH 5.5 as determined by SEC after four or eight weeks of thermal stress at 40° C. Additionally, the increase in the percent HMW aggregate products in the stable aqueous composition comprising 10 mg/ml of anti-C5 antibody (e.g., 10 mg/ml eculizumab formulated with the stabilizer trehalose, sucrose, sorbitol, arginine, or a combination thereof) with pH of about 5.5 can be reduced by about 5% or more (e.g., about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 98% or more, about 99% or more, about 100% or a range between any of these values) or by about 5% to about 100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, about 100% or a range between any of these values) as compared to a composition containing 10 mg/ml of the antibody formulated in 10 mM histidine buffer, 150 mM sodium chloride, 0.022% polysorbate 80, pH 5.5 as determined by SEC after five cycles of freeze/thaw (−70° C./RT).

When the stable aqueous composition contains 10 mg/ml of an anti-C5 antibody with pH of about 7.0 (e.g., 10 mg/ml eculizumab formulated with the stabilizer trehalose, sucrose, sorbitol, arginine, or a combination thereof), the composition also has increased stability as compared to an anti-C5 antibody in the same amount (10 mg/ml) formulated in 10 mM phosphate buffer, 150 mM sodium chloride, 0.022% polysorbate 80, pH 7.0 (i.e., the SOLIRIS™ eculizumab formulation). The stability can be measured by any suitable means, for example, the increase in the percent of HMW aggregate products as determined by SEC (1) after four or eight weeks of thermal stress at 40° C. or (2) after five cycles of freeze/thaw (−70° C./RT). Additionally, the stability can be measured by the increase in acidic content as determined anion-exchange high performance liquid chromatography (AEX-HPLC) after four or eight weeks of thermal stress at 40° C.

Accordingly, the invention also provides a method of analyzing HMW aggregate products by performing SEC at 0, 1, 2, 4, and/or 8 weeks of thermal stress at 40° C. or at 0, 1, 3, and/or 5 cycles of freeze/thaw (−70° C./RT) and determining the change in HMW aggregate products. The invention also provides a method of analyzing the acidic content by performing AEX-HPLC at 0, 1, 2, 4 and/or 8 weeks of thermal stress at 40° C. and determining the change in acidic content.

In comparison with a composition containing 10 mg/ml of the antibody formulated in 10 mM phosphate buffer, 150 mM sodium chloride, 0.022% polysorbate 80, pH 7.0 (i.e., the SOLIRIS™ eculizumab formulation), the stable aqueous composition comprising 10 mg/ml of anti-C5 antibody (e.g., 10 mg/ml eculizumab formulated with the stabilizer trehalose, sucrose, sorbitol, arginine, or a combination thereof) with a pH of about 7.0 has less of an increase in the percent of HMW aggregate products as determined by SEC (1) after four or eight weeks of thermal stress at 40° C. or (2) after five cycles of freeze/thaw (−70° C./RT). For example, the increase in the percent HMW aggregate products in the stable aqueous composition comprising 10 mg/ml of anti-C5 antibody (e.g., 10 mg/ml eculizumab formulated with the stabilizer trehalose, sucrose, sorbitol, arginine, or a combination thereof) with pH of about 7.0 can be reduced by about 2% or more (e.g., about 2% or more, about 5% or more, about 10% or more, about 15% or more, about 20% or more, about 25% or more, about 28% or more, about 29% or more, about 30% or more, about 35% or more, about 38% or more about 39% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or a range between any of these values), or by about 2% to about 50% (e.g., about 2%, about 3%, about 5%, about 10%, about 15%, about 20%, about 25%, about 28%, about 29%, about 30%, about 35%, about 38%, about 39%, about 40%, about 45%, about 50%, or a range between any of these values) as compared to a composition containing 10 mg/ml of the antibody formulated in 10 mM phosphate buffer, 150 mM sodium chloride, 0.022% polysorbate 80, pH 7.0 (i.e., the SOLIRIS™ eculizumab formulation) as determined by SEC after four or eight weeks of thermal stress at 40° C. For example, the increase in the percent HMW aggregate products in the stable aqueous composition comprising 10 mg/ml of anti-C5 antibody (e.g., 10 mg/ml eculizumab formulated with the stabilizer trehalose, sucrose, sorbitol, arginine, or a combination thereof) with a pH of about 7.0 can be reduced by about 5% or more (e.g., about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 98% or more, about 99% or more, about 100% or a range between any of these values), or by about 5% to about 100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, about 100% or a range between any of these values) as compared to a composition containing 10 mg/ml of the antibody formulated in 10 mM phosphate buffer, 150 mM sodium chloride, 0.022% polysorbate 80, pH 7.0 (i.e., the SOLIRIS™ eculizumab formulation) as determined by SEC after five cycles of freeze/thaw (−70° C./RT).

In comparison with a composition containing 10 mg/ml of the antibody formulated in 10 mM phosphate buffer, 150 mM sodium chloride, 0.022% polysorbate 80, pH 7.0 (i.e., the SOLIRIS™ eculizumab formulation), the stable aqueous composition comprising 10 mg/ml of anti-C5 antibody (e.g., 10 mg/ml eculizumab formulated with the stabilizer trehalose, sucrose, sorbitol, arginine, or a combination thereof) with a pH of about 7.0 has less of an increase in acidic content percent as determined by AEX-HPLC after four or eight weeks of thermal stress at 40° C. For example, the increase in the acidic content percent in the stable aqueous composition can be reduced by about 10% or more (e.g., about 10% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 76% or more, about 80% or more, about 85% or more, about 86% or more, about 87% or more, about 90% or more, about 95% or more, about 96% or more, about 97% or more, about 100%, or a range between any of these values) or by about 10% to about 100% (e.g., about 10%, about 12%, about 13%, about 14%, about 20%, about 25%, about 30%, about 35%, about 40%, about 45%, about 50%, about 55%, about 60%, about 65%, about 70%, about 75%, about 76%, about 80%, about 85%, about 86%, about 87%, about 90%, about 95%, about 96%, about 97%, about 100%, or a range between any of these values) as compared to a composition containing 10 mg/ml of the antibody formulated in 10 mM phosphate buffer, 150 mM sodium chloride, 0.022% polysorbate 80, pH 7.0 (i.e., the SOLIRIS™ eculizumab formulation) as determined by AEX-HPLC after four or eight weeks of thermal stress at 40° C.

When the stable aqueous composition contains a high concentration of anti-C5 antibody (e.g., 50-100 mg/ml) (i.e., a concentrated composition useful for storage of an anti-C5 antibody), the composition has increased stability as compared to a composition containing a high concentration (e.g., 50-100 mg/ml of an anti-C5 antibody) formulated in 10 mM phosphate buffer, 150 mM sodium chloride, 0.022% polysorbate 80, pH 7.0. The stability can be measured by any suitable means, for example, the increase in the percent of HMW aggregate products as determined by SEC (1) after four weeks of thermal stress at 25° C. or (2) after five cycles of freeze/thaw (−70° C./RT). Additionally, the stability can be measured by the increase in acidic content as determined anion-exchange high performance liquid chromatography (AEX-HPLC) after four weeks of thermal stress at 25° C.

Accordingly, the invention provides a method of analyzing HMW aggregate products by performing SEC after at 0, 1, 2, and/or 4 weeks of thermal stress at 25° C. or at 0, 1, 3, and/or 5 cycles of freeze/thaw (−70° C./RT) and determining the change in HMW aggregate products. The invention also provides a method of analyzing the acidic content by performing AEX-HPLC at 0, 1, 2, and/or 4 weeks of thermal stress at 25° C. and determining the change in acidic content.

In comparison with a composition containing a high concentration of anti-C5 antibody (e.g., 50-100 mg/ml of an anti-C5 antibody) formulated in 10 mM phosphate buffer, 150 mM sodium chloride, 0.022% polysorbate 80, pH 7.0, the stable aqueous composition comprising a high concentration of anti-C5 antibody (e.g., 50-100 mg/ml eculizumab formulated with the stabilizer trehalose, sucrose, sorbitol, arginine, or a combination thereof) has less of an increase in the percent of HMW aggregate products as determined by SEC (1) after four weeks of thermal stress at 25° C. or (2) after five cycles of freeze/thaw (−70° C./RT). For example, the increase in the percent HMW aggregate products in the stable aqueous composition comprising a high concentration of anti-C5 antibody (e.g., 50-100 mg/ml eculizumab formulated with the stabilizer trehalose, sucrose, sorbitol, arginine, or a combination thereof) can be reduced by about 3% or more (e.g., about 3% or more, about 4% or more, about 5% or more, about 6% or more, about 7% or more, about 8% or more, about 9% or more, about 10% or more, about 15% or more, about 20% or more, about 25% or more about 30% or more, about 35% or more, about 40% or more, about 45% or more, about 50% or more, about 55% or more, about 60% or more, about 65% or more, about 70% or more, about 75% or more, about 77% or more, about 78% or more, about 79% or more, about 80% or more, about 85% or more, about 90% or more, about 95% or more, about 100%, or a range between any of these values) or by about 3% to about 90% (e.g., about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 77%, about 78%, about 79%, about 80%, about 85%, about 90%, about 95%, about 100%, or a range between any of these values) as compared to a composition containing the same amount of antibody (i.e., a high concentration , e.g., 50-100 mg/ml, of the antibody) formulated in 10 mM phosphate buffer, 150 mM sodium chloride, 0.022% polysorbate 80, pH 7.0 as determined by SEC after four weeks of thermal stress at 25° C.

In comparison with a composition containing a high concentration (e.g., 50-100 mg/ml) of anti-C5 antibody formulated in 10 mM phosphate buffer, 150 mM sodium chloride, 0.022% polysorbate 80, pH 7.0, the stable aqueous composition comprising a high concentration of anti-C5 antibody (e.g., 50-100 mg/ml eculizumab formulated with the stabilizer trehalose, sucrose, sorbitol, arginine, or a combination thereof) has less of an increase in acidic content percent as determined by AEX-HPLC after four weeks of thermal stress at 25° C. For example, the increase in the acidic content percent in the stable aqueous composition can be reduced by about 10% or more (e.g., about 10% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 76% or more, about 80% or more, about 85% or more, about 86% or more, about 87% or more, about 90% or more, about 95% or more, about 96% or more, about 97% or more, about 100%, or a range between any of these values) or by about 10% to about 100% (e.g., about 10%, about 12%, about 13%, about 14%, about 20%, about 25%, about 30%, about 35%, about 40%, about 45%, about 50%, about 55%, about 60%, about 65%, about 70%, about 75%, about 76%, about 80%, about 85%, about 86%, about 87%, about 90%, about 95%, about 96%, about 97%, about 100%, or a range between any of these values) as compared to a composition containing a high concentration (e.g., 50-100 mg/ml) of the antibody formulated in 10 mM phosphate buffer, 150 mM sodium chloride, 0.022% polysorbate 80, pH 7.0 as determined by AEX-HPLC after four weeks of thermal stress at 25° C.

In a particular embodiment, the stable aqueous composition consists essentially of (a) about 10 mg/ml of an anti-C5 antibody, (b) about 0.01 to about 0.1% (w/v) of a surfactant, (c) about 1 to about 20 mM buffer having a pH of 5.5 to 7.5, and (d) a stabilizer selected from the group consisting of trehalose, sucrose, sorbitol, arginine, or a combination thereof. Preferably, the surfactant comprises 0.022% (w/v) polysorbate 80, the stabilizer is arginine or trehalose (e.g., 150 mM arginine or 9.5% trehalose), and anti-C5 antibody is eculizumab. Such a composition (e.g., pharmaceutical composition or pharmaceutical formulation) is suitable for administration, e.g., intravenous administration, to a subject.

In a particular embodiment, the stable aqueous composition consists essentially of (a) about 50 mg/ml of an anti-C5 antibody, (b) about 0.01 to about 0.1% (w/v) of a surfactant, (c) about 1 to about 20 mM buffer having a pH of 5.5 to 7.5, and (d) a stabilizer selected from the group consisting of trehalose, sucrose, sorbitol, arginine, or a combination thereof. Preferably, the surfactant comprises 0.022% (w/v) polysorbate 80, the stabilizer is arginine or trehalose (e.g., 150 mM arginine or 9.5% trehalose), and anti-C5 antibody is eculizumab. Such a composition is suitable for storage and can be diluted prior to administration (e.g., intravenous administration) to a subject.

In addition to the above-described components, the stable aqueous composition can contain excipients that inhibit adsorption, prevent oxidation, maintain pH, stabilize the anti-C5 antibody, and control the osmolality of the composition. Excipients can be chosen on the basis of the mechanisms by which they stabilize proteins against various chemical and physical stresses that could occur during a manufacturing process, under particular storage conditions, or associated with a particular mode of administration. In addition, an excipient can function as a diluent or employed to reduce the viscosity in high protein formulations in order to enable the delivery and/or enhance patient convenience.

The concentration or amount of an excipient to use in the stable aqueous composition varies depending on, for example, the amount of anti-C5 antibody included in the composition, the amount of other excipients included in the composition, whether a diluent is needed, the amount or volume of other components in the composition, and the tonicity or osmolality that is desired to be achieved. In various embodiments, different types of excipients can be combined. Accordingly, the composition can contain one, two, three, or more different types of excipients. Those skilled in the art can determine what amount or concentration of excipient can be included.

Salts may be used to adjust the ionic strength and/or the isotonicity of the stable aqueous composition and/or to improve the physical stability of the anti-C5 antibody or other ingredients of the composition. Salts can prevent or reduce protein insolubility and/or aggregation, and reduce the viscosity of protein formulations.

The stable aqueous composition can be isotonic. The tonicity of the composition can be controlled by any suitable means including the addition of a tonicity-adjusting agent. Suitable tonicity-adjusting agents include, but are not limited to, dextrose, glycerin, mannitol, potassium chloride, and sodium chloride.

The stable aqueous composition can have any suitable osmolality. For example, the composition can have an osmolality of about 200 to about 400 (e.g., about 200, about 210, about 220, about 230, about 240, about 250, about 260, about 270, about 280, about 290, about 300, about 310, about 320, about 330, about 340, about 350, about 360, about 370, about 380, about 390, about 400, or a range between any of these values) mOsm/kg. In a particular embodiment, the composition has an osmolality of about 300 mOsm/kg.

The stable aqueous composition can have any suitable viscosity. For example, the viscosity of the composition can be less than about 50 (e.g., less than about 45, less than about 40, less than about 35, less than about 30, less than about 25, less than about 20, less than about 15, less than about 10, less, less than about 5, or less than about 1) cP at 25° C. In a preferred embodiment, the viscosity of the composition can be less than about 20 (e.g., less than about 18, less than about 15, less than about 12, less than about 10, less than about 8, less than about 5, less than about 3, or less than about 1) cP at 25° C. In an even more preferred embodiment, the viscosity of the composition can be less than about 10 (e.g., less than about 9, less than about 8, less than about 7, less than about 6, less than about 5, less than about 4, less than about 3, less than about 2, or less than about 1) cP at 25° C.

The stable aqueous composition can have any suitable conductivity. For example, the conductivity of the composition can be less than about 20 (e.g., less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2, or less than about 1) mS/cm.

The anti-C5 antibody in the stable aqueous composition neutralizes human C5 activity with an IC₅₀ of about 2.0-4.0 μg/mL (e.g., about 2.0 μg/mL, about 2.5 μg/mL, about 2.7 μg/mL, about 2.8 μg/mL, about 2.9 μg/mL, about 3.0 μg/mL, about 3.5 μg/mL, about 4.0 μg/mL, or a range between any of these values) or about 2.0-3.5 μg/mL (e.g., about 2.0 μg/mL, about 2.5 μg/mL, about 2.7 μg/mL, about 2.8 μg/mL, about 2.9 μg/mL, about 3.0 μg/mL, about 3.5 μg/mL, or a range between any of these values) based on an in vitro hemolysis assay. The in vitro hemolysis assay evaluates red blood cell lysis in a sample containing red blood cells (e.g., plasma) following exposure to the composition. The in vitro hemolysis assay can comprise (a) diluting the composition (e.g., with Dextrose-Gelatin-Veronal (DGV) solution; Lonza Cat. No. 10-539B), (b) adding human serum (e.g., 9.6% human serum) to serially diluted compositions (e.g., 0.8-13.5 μg/ml) and incubating at room temperature (e.g., for 30±5 minutes), and (c) measuring hemolysis. In a particular embodiment, the diluted composition can be added to a solution containing chicken red blood cells (e.g., 22.3×10⁵ cells/well) and incubated for 20±5 minutes at 37° C. in a 5% CO2 incubator. Cytotox-Glo™ reagent can be added and the resulting composition is incubated 25° C., 300 rpm for 15 minutes after which luminescence is measured as an indicator of hemolysis.

The composition is suitable for administration to a subject by any suitable mode of administration including, but not limited to, oral, aerosol, parenteral (e.g., subcutaneous, intravenous, intra-arterial, intramuscular, intradermal, intraperitoneal, intracerebrospinal, intrasynovial, and intrathecal), rectal, and vaginal administration. Parenteral administration can be by bolus injection or continuous infusion. Compositions for injection may be presented in unit dosage form, e.g., in ampoules or in multi-dose containers.

In one embodiment, the composition is suitable for parental administration and is packaged in a pre-filled syringe. In a particular example, the composition is suitable for intravenous injection.

In another embodiment, the composition is formulated as a depot preparation. Such long acting compositions may be administered by implantation (for example subcutaneously or intramuscularly) or by intramuscular injection. Thus, for example, the composition can be modified with suitable polymeric or hydrophobic materials (e.g., as an emulsion in an acceptable oil) or ion exchange resins, or as sparingly soluble derivatives, for example, as a sparingly soluble salt.

In yet another embodiment, the composition is presented in a vial, pre-filled syringe, or device that contains one or more unit dosage forms containing the anti-C5 antibody. The device can comprise a syringe having a single dose of the liquid composition ready for injection. The syringe can be accompanied by instructions for administration. The device can contain a cartridge. The invention also provides a kit or container that comprises the composition. The kit also can be accompanied by instructions for use.

The subject to be administered the composition can be any suitable subject. The subject can be a mammal, such as a mouse, rat, guinea pig, hamster, rabbit, cat, dog, pig, cow, horse, or primate (e.g., human) In one embodiment, the subject has, or is at risk for having, a disorder in which C5 activity is detrimental.

In this regard, the invention provides a method of treating a disorder in which C5 activity is detrimental in a subject comprising administering the composition (e.g., a therapeutically effective amount of the composition) to the subject.

The disorder in which C5 activity is detrimental in a subject includes, but is not limited to, hemolytic disease, paroxysmal nocturnal hemoglobinuria (PNH), atypical hemolytic uremic syndrome (aHUS), dermatomyositis, idiopathic membranous glomerular nephropathy, acute humoral rejection (AHR, also known as antibody-mediated rejection (AMR)) such as for kidney allografts or renal transplantation, myasthenia gravis, neuromyelitis optica, membranoproliferative glomerulonephritis (MPGN), dense-deposit disease (DDD), cold agglutinin disease, catastrophic antiphospholipid syndrome (CAPS), and shiga-toxin-producing Escherichia coli hemolytic-uremic syndrome (STEC-HUS).

In a first embodiment, the disorder to be treated is selected from the group consisting of hemolytic disease, PNH, and aHUS. In a second embodiment, the disorder to be treated is hemolytic disease. In a third embodiment, the disorder to be treated is PNH. In a fourth embodiment, the disorder to be treated is aHUS.

The term “treating” refers to administration or application of remedies for a disorder in a subject and includes inhibiting the disorder, arresting development of the disorder, relieving the disorder (for example, by causing regression, or restoring or repairing a lost, missing, or defective function) or stimulating an inefficient process. The term includes obtaining a desired pharmacologic and/or physiologic effect and covering any treatment of a pathological condition or disorder in a subject. The term encompasses a therapeutic effect in terms of a partial or complete cure for a disorder and/or adverse effect attributable to the disorder. Treating includes inhibiting the disorder, such as arresting its development, stopping or terminating the disorder or at least its associated symptoms, so that the subject no longer suffers from the disorder or its symptoms, such as causing regression of the disorder or its symptoms, for example, by restoring or repairing a lost, missing or defective function, or stimulating an inefficient process, or relieving, alleviating or ameliorating the disorder, or symptoms associated therewith, where ameliorating is used in a broad sense to refer to at least a reduction in the magnitude of a parameter, such as inflammation, pain and/or tumor size.

Administration of the composition to a subject can also be prophylactic. The term “preventing” encompasses complete or partial prevention of a disorder or symptom thereof, i.e., preventing the disorder from occurring or recurring in a subject who may be predisposed to the disorder but is not yet symptomatic.

The appropriate dosage, or therapeutically effective amount, of the anti-C5 antibody will depend on the condition to be treated, the severity of the condition, prior therapy, and the subject's clinical history and response to the anti-C5 antibody. The proper dose can be adjusted according to the judgment of the attending physician such that it can be administered to the subject one time or over a series of administrations. The composition can be administered as a sole therapeutic or in combination with additional therapies as needed.

In certain embodiments, an acceptable dose for administration by injection contains about 300-1200 mg/dose (e.g., about 300 mg/dose, about 400 mg/dose, about 500 mg/dose, about 600 mg/dose, about 700 mg/dose, about 800 mg/dose, about 900 mg/dose, about 1000 mg/dose, about 1100 mg/dose, about 1200 mg/dose, or a range between any of these values). The dose can be administered at weekly doses or separated by several weeks (for example, 2 to 8 weeks, e.g., 2 weeks, 3 weeks, 4 weeks, 5 weeks, 6 weeks, 7 weeks, or 8 weeks).

In some instances, an improvement in a subject's disorder can be obtained by a dose of up to about 1200 mg of the composition per week over a period of at least five weeks, though treatment for longer periods may be necessary to induce the desired degree of improvement. For incurable chronic conditions the regimen may be continued indefinitely.

In one embodiment, the disorder to be treated is PNH, and the subject is administered 600 mg of the anti-C5 antibody once per week for four weeks for a total of four doses, a fifth dose of 900 mg of the anti-C5 antibody one week following the fourth dose, and 900 mg of the anti-C5 antibody every two weeks following the fifth dose.

In another embodiment, the disorder to be treated is aHUS, and the subject is administered 900 mg of the anti-C5 antibody once per week for four weeks for a total of four doses, a fifth dose of 1200 mg of the anti-C5 antibody one week following the fourth dose, and 1200 mg of the anti-C5 antibody every two weeks following the fifth dose.

The composition can be diluted to reduce the concentration of the anti-C5 antibody prior to administering to a subject. For example, the concentration of the anti-C5 antibody in the composition to be administered to the subject (e.g., infused) can be about 5 mg/ml. Excipients in the composition to be administered to the subject can include, for example, sodium phosphate monobasic, sodium phosphate dibasic, sodium chloride, polysorbate (e.g., polysorbate 80), and water.

The composition can be administered to the subject alone or in combination (e.g., sequential or simultaneous administration) with another active agent or treatment. Examples include, but are not limited to, plasmapheresis, immunosuppressive therapy, and intravenous immunoglobulin.

The following examples further illustrate the invention but, of course, should not be construed as in any way limiting its scope.

### EXAMPLES

### Example 1

This example demonstrates the stabilizing effect of particular stabilizers against stress conditions at pH 5.5 and 7.0.

To test the stability of formulations of anti-C5 antibody (10 mg/ml eculizumab) compared to conventional eculizumab formulation (i.e. SOLIRIS™ eculizumab formulation), multiple different formulations were prepared including seven different stabilizer candidates (NaCl, trehalose, sucrose, sorbitol, mannitol, arginine, and glycine) and two types of buffer solutions (pH 5.5 histidine buffer and pH 7.0 sodium phosphate buffer).

TABLE 1
SOLIRIS ™ Eculizumab Formulation Information

| | |
|---|---|
| Ab | 10 mg/ml; 30 ml |
| Concentration/ | |
| Volume | |
| Formulation | 150 mM NaCl |
| | 10 mM sodium phosphate buffer (1.78 mg/ml sodium |
| | phosphate dibasic, 0.46 mg/ml sodium phosphate |
| | monobasic) |
| | 0.022% polysorbate 80 |
| | pH 7.0 |

TABLE 2
Sample Formulation Components

| Sample | Antibody | | | | |
|---|---|---|---|---|---|
| Number | Concentration | pH | Buffer | Stabilizer | Surfactant |
| 1 | 10 mg/ml | 5.5 | 10 mM | 150 mM NaCl | 0.022% |
| 2 | | | histidine | 9.5% trehalose | polysorbate |
| 3 | | | | | 80 |
| 4 | | | | | |
| 5 | | | | 8.5% sucrose | |
| 6 | | | | 5.0% sorbitol | |
| 7 | | | | 150 mM | |
| | | | | arginine | |
| 8 | | 7.0 | 10 mM | 150 mM NaCl | |
| 9 | | | sodium | | |
| 10 | | | phosphate | | |
| 11 | | | | 9.5% trehalose | |
| 12 | | | | | |
| 13 | | | | | |
| 14 | | | | 8.5% sucrose | |
| 15 | | | | 5.0% sorbitol | |
| 16 | | | | 150 mM | |
| | | | | arginine | |

Each of the samples was exposed to experimentally controlled stresses: (1) 0, 1, 2, or 4 weeks of 40° C. thermal stress and (2) 1, 3, or 5 cycles of freeze/thaw (−70° C./RT). Afterwards, samples were collected to be analyzed and evaluated for their resistance to stress-induced product quality change as a function of high molecular weight percent (HMW %) measurements (SE-HPLC).

TABLE 3
ΔHMW % Summary of Samples at 0, 1, 2 and 4 Weeks (pH 5.5)

| | |
|---|---|
| | 40° C., |
| ΔHMW % | Thermal Stress Condition |

| Sample No. | Stabilizer | 0 wk | 1 wk | 2 wk | 4 wk |
|---|---|---|---|---|---|
| 1 | NaCl | 0.00 | 0.30 | 0.83 | 2.36 |
| 2 | Trehalose | 0.00 | 0.23 | 0.56 | 0.94 |
| 3 | Trehalose | 0.00 | 0.21 | 0.44 | 1.03 |
| 4 | Trehalose | 0.00 | 0.24 | 0.51 | 0.99 |
| 5 | Sucrose | 0.00 | 0.29 | 0.42 | 0.98 |
| 6 | Sorbitol | 0.00 | −0.01 | 0.09 | 0.38 |
| 7 | Arginine | 0.00 | 0.28 | 0.57 | 1.26 |

In samples in pH 5.5 histidine buffer exposed to the thermal stress condition (40° C.), the degree of HMW % increase was as follows: sorbitol had a 0.4% increase, followed by trehalose (ΔHMW %=1.0%), sucrose (ΔHMW %=1.0%), arginine (ΔHMW %=1.3%) and NaCl (ΔHMW %=2.4%). In samples in pH 7.0 prepared with the same formulation as the SOLIRIS™ eculizumab formulation (Sample Nos. 8-10) exposed to the thermal stress condition (40° C.), the degree of HMW % increase was 1.97% (average).

The increase in the percent HMW aggregate products in the stable aqueous composition comprising a 10 mg/ml of anti-C5 antibody can be reduced by about 5% or more, or by about 5% to about 100% (e.g., about 5%, about 10%, about 20%, about 30%, about 40%, about 50%, about 60%, about 70%, about 80%, about 88%, about 89%, about 90%, about 91%, about 92%, about 95%, about 97%, about 98%, about 99%, about 100%, or a range between any of these values) as compared to a composition containing 10 mg/ml of the antibody formulated in 10 mM phosphate buffer, 150 mM sodium chloride, 0.022% polysorbate 80, pH 7.0 (i.e., SOLIRIS™ eculizumab formulation) as determined by SEC after four weeks of thermal stress at 40° C.

TABLE 4
ΔHMW % Summary of Samples after 0, 1, 3 and
5 F/T Cycles (pH 5.5)

| | |
|---|---|
| ΔHMW % | Freeze/Thaw Cycling |

| Sample No. | Stabilizer | 0 | 1 | 3 | 5 |
|---|---|---|---|---|---|
| 1 | NaCl | 0.00 | 1.12 | 2.55 | 5.55 |
| 2 | Trehalose | 0.00 | 0.01 | 0.02 | 0.03 |
| 3 | Trehalose | 0.00 | −0.01 | 0.02 | 0.01 |
| 4 | Trehalose | 0.00 | −0.01 | 0.02 | 0.02 |
| 5 | Sucrose | 0.00 | 0.00 | 0.01 | 0.03 |
| 6 | Sorbitol | 0.00 | 0.00 | 0.00 | 0.01 |
| 7 | Arginine | 0.00 | 0.01 | 0.03 | 0.07 |

For freeze-thaw cycling-induced stress, trehalose, sucrose, sorbitol, and arginine in pH 5.5 histidine buffer successfully hindered formation of protein aggregates (which is detected as the HMW portion in a sample) during freeze-thaw cycling. Even after five freeze-thaw cycles, HMW % increase at pH 5.5 was less than 0.1%. as opposed to the 5.55% increase observed in samples prepared with a formulation comprising NaCl, which is the stabilizer in the SOLIRIS™ eculizumab formulation (Sample No. 1) and 11.38% increase (average) observed in samples at pH 7.0 prepared with the same formulation as the SOLIRIS™ eculizumab formulation (Sample Nos. 8-10).

The increase in the percent HMW aggregate products in the stable aqueous composition comprising a 10 mg/ml of anti-C5 antibody can be reduced by about 5% or more, or by about 5% to about 100% (e.g., about 5%, about 10%, about 20%, about 30%, about 40%, about 50%, about 60%, about 70%, about 80%, about 90%, about 95%, about 99%, about 100%, or a range between any of these values) as compared to a composition containing 10 mg/ml of the antibody formulated in 10 mM phosphate buffer, 150 mM sodium chloride, 0.022% polysorbate 80, pH 7.0 (i.e., SOLIRIS™ eculizumab formulation) as determined by SEC after five cycles of freeze/thaw (−70° C./RT).

TABLE 5
ΔHMW % Summary of Samples at 0, 1, 2 and 4 Weeks (pH 7.0)

| | |
|---|---|
| | 40° C., |
| ΔHMW % | Thermal Stress Condition |

| Sample No. | Stabilizer | 0 wk | 1 wk | 2 wk | 4 wk |
|---|---|---|---|---|---|
| 8 | NaCl | 0.00 | 0.99 | 1.33 | 2.02 |
| 9 | NaCl | 0.00 | 0.98 | 1.34 | 1.90 |
| 10 | NaCl | 0.00 | 0.98 | 1.48 | 1.99 |
| 11 | Trehalose | 0.00 | 0.69 | 0.89 | 1.57 |
| 12 | Trehalose | 0.00 | 0.70 | 0.99 | 1.47 |
| 13 | Trehalose | 0.00 | 0.69 | 0.98 | 1.40 |
| 14 | Sucrose | 0.00 | 0.72 | 0.97 | 1.40 |
| 15 | Sorbitol | 0.00 | 0.78 | 1.10 | 1.93 |
| 16 | Arginine | 0.00 | 0.40 | 0.63 | 1.22 |

In samples in pH 7.0 sodium phosphate buffer, the sample containing arginine showed stabilizing capacity with a ΔHMW % of 1.2% followed by sucrose, trehalose, and sorbitol with 1.4%, 1.5%, and 1.9% increases, respectively.

TABLE 6
ΔHMW % Summary of Samples after 0, 1, 3 and
5 F/T Cycles (pH 7.0)

| | |
|---|---|
| ΔHMW % | Freeze/Thaw Cycling |

| Sample No. | Stabilizer | 0 | 1 | 3 | 5 |
|---|---|---|---|---|---|
| 8 | NaCl | 0.00 | 1.49 | 6.13 | 9.71 |
| 9 | NaCl | 0.00 | 2.69 | 7.85 | 12.14 |
| 10 | NaCl | 0.00 | 2.97 | 8.20 | 12.28 |
| 11 | Trehalose | 0.00 | −0.01 | 0.00 | −0.02 |
| 12 | Trehalose | 0.00 | 0.00 | 0.05 | 0.03 |
| 13 | Trehalose | 0.00 | 0.02 | 0.06 | 0.05 |
| 14 | Sucrose | 0.00 | 0.00 | 0.04 | 0.04 |
| 15 | Sorbitol | 0.00 | −0.01 | 0.03 | 0.04 |
| 16 | Arginine | 0.00 | 0.03 | 0.03 | 0.06 |

For freeze-thaw cycling-induced stress, trehalose, sucrose, sorbitol, and arginine in pH 7.0 phosphate buffer successfully hindered formation of protein aggregates (which is detected as the HMW portion in a sample) during freeze-thaw cycling. Even after five freeze-thaw cycles, HMW % increase at pH 7.0 was less than 0.1% as opposed to 11.38% average increase observed in samples prepared with the same formulation as the SOLIRIS™ eculizumab formulation (Sample Nos. 8-10).

Accordingly, trehalose, sucrose, sorbitol, and arginine possess superior stabilizing capability as compared to NaCl, which is the stabilizer in the SOLIRIS™ eculizumab formulation.

### Example 2

This example confirms the stabilizing effect of several stabilizers against stress conditions at pH 7.0.

To test the stability of formulations of anti-C5 antibody (10 mg/ml eculizumab) compared to conventional eculizumab formulation (i.e. SOLIRIS™ eculizumab formulation), formulations were prepared including 3 different stabilizer candidates (NaCl, trehalose, and arginine).

TABLE 7
Sample Formulation Components

| Sample | Antibody | | | | |
|---|---|---|---|---|---|
| Number | Concentration | pH | Buffer | Stabilizer | Surfactant |
| 1 | 10 mg/ml | 7.0 | 10 mM | 150 mM NaCl | 0.022% |
| 2 | | | sodium | | polysorbate |
| 3 | | | phosphate | | 80 |
| 4 | | | | 9.5% trehalose | |
| 5 | | | | | |
| 6 | | | | | |
| 7 | | | | 150 mM | |
| 8 | | | | arginine | |
| 9 | | | | | |

Each of the samples was exposed to experimentally controlled stresses: (1) 0, 1, 2, 4 or 8 weeks of 40° C. thermal stress and (2) 1, 3, or 5 cycles of freeze/thaw (−70° C./RT). Afterwards, samples were collected to be analyzed and evaluated for their resistance to stress-induced product quality change as a function of high molecular weight percent (HMW %) measurements (SEC) and acidic percent (Acidic %) measurements (AEX-HPLC).

TABLE 8
ΔHMW % and ΔAcidic % Summary of Samples at 0, 1, 2, 4 and 8 Weeks

| | | 40° C., Thermal Stress Condition | | | | | | | | | |
|---|---|---|---|---|---|---|---|---|---|---|---|
| | | 0 wk | | 1 wk | | 2 wk | | 4 wk | | 8 wk | |
| No. | Stabilizer | ΔHMW % | ΔAcidic % | ΔHMW % | ΔAcidic % | ΔHMW % | ΔAcidic % | ΔHMW % | ΔAcidic % | ΔHMW % | ΔAcidic % |
| 1 | NaCl | 0.00 | 0.00 | 0.43 | 5.99 | 0.76 | 11.45 | 1.31 | 22.17 | 4.27 | 35.09 |
| 2 | NaCl | 0.00 | 0.00 | 0.47 | 6.10 | 0.77 | 11.47 | 1.25 | 22.28 | 3.89 | 35.09 |
| 3 | NaCl | 0.00 | 0.00 | 0.50 | 5.96 | 0.79 | 10.70 | 1.40 | 21.70 | 3.11 | 34.39 |
| 4 | Trehalose | 0.00 | 0.00 | 0.34 | 5.11 | 0.53 | 10.89 | 1.04 | 20.54 | 2.38 | 32.57 |
| 5 | Trehalose | 0.00 | 0.00 | 0.34 | 5.57 | 0.52 | 10.85 | 0.79 | 20.53 | 2.09 | 32.37 |
| 6 | Trehalose | 0.00 | 0.00 | 0.30 | 5.38 | 0.48 | 10.65 | 1.03 | 20.64 | 1.50 | 31.55 |
| 7 | Arginine | 0.00 | 0.00 | 0.32 | 6.38 | 0.58 | 12.35 | 1.15 | 23.34 | 4.12 | 36.81 |
| 8 | Arginine | 0.00 | 0.00 | 0.29 | 6.84 | 0.56 | 12.86 | 1.24 | 23.81 | 4.37 | 37.28 |
| 9 | Arginine | 0.00 | 0.00 | 0.27 | 6.44 | 0.44 | 12.73 | 1.51 | 23.48 | 4.25 | 37.39 |

In samples in pH 7.0 phosphate buffer exposed to the thermal stress condition (40° C.), the degree of HMW % increase and the degree of acidic % increase were as follows: trehalose had ΔHMW % of 2.0% and ΔAcidic % of 32.2% increases, and arginine had ΔHMW % of 4.2% and ΔAcidic % of 37.2% increases.

TABLE 9
ΔHMW % and ΔAcidic % Summary of Samples after 0, 1, 3 and 5 F/T Cycles

| | | Freeze/Thaw Cycling | | | | | | | |
|---|---|---|---|---|---|---|---|---|---|
| | | 0 cycle | | 1 cycle | | 3 cycle | | 5 cycle | |
| No. | Stabilizer | ΔHMW % | ΔAcidic % | ΔHMW % | ΔAcidic % | ΔHMW % | ΔAcidic % | ΔHMW % | ΔAcidic % |
| 1 | NaCl | 0.00 | 0.00 | 1.94 | −0.76 | 6.56 | −1.24 | 10.52 | 0.22 |
| 2 | NaCl | 0.00 | 0.00 | 2.19 | 0.17 | 6.42 | −0.70 | 9.20 | 0.24 |
| 3 | NaCl | 0.00 | 0.00 | 2.47 | −0.89 | 6.97 | −1.95 | 9.94 | −0.58 |
| 4 | Trehalose | 0.00 | 0.00 | −0.03 | 0.37 | 0.03 | −0.08 | 0.11 | 0.51 |
| 5 | Trehalose | 0.00 | 0.00 | 0.00 | 0.28 | 0.02 | −0.38 | 0.08 | 0.77 |
| 6 | Trehalose | 0.00 | 0.00 | −0.01 | 0.24 | 0.00 | 0.50 | 0.05 | 0.47 |
| 7 | Arginine | 0.00 | 0.00 | −0.04 | 0.49 | 0.02 | 0.29 | 0.05 | 0.59 |
| 8 | Arginine | 0.00 | 0.00 | −0.01 | 0.37 | 0.00 | 0.39 | −0.05 | 0.78 |
| 9 | Arginine | 0.00 | 0.00 | −0.01 | 0.17 | 0.00 | 0.08 | −0.04 | 0.34 |

For freeze-thaw cycling-induced stress, the degree of HMW % increase was as follows: trehalose with ΔHMW % of 0.1% and arginine with ΔHMW % of 0.0% , respectively

### Example 3

This example provides a feasibility study for pharmaceutical compositions comprising a high concentration of anti-C5 antibody.

To test the stability and feasibility of higher concentration of formulations of anti-C5 antibody (30 or 50 mg/ml) compared to conventional eculizumab formulation (i.e. SOLIRIS™ eculizumab formulation) or a formulation comprising NaCl, which is the stabilizer used in the SOLIRIS™ eculizumab formulation, multiple different formulations were prepared including five different stabilizer candidates (NaCl, trehalose, arginine, sorbitol, and sucrose).

TABLE 10
Sample Formulation Components

| | Antibody | | | | |
|---|---|---|---|---|---|
| Sample | Concentration | | | | |
| Number | (mg/ml) | pH | Buffer | Stabilizer | Surfactant |
| 1 | 30 | 7.0 | 10 mM | 150 mM | 0.022% |
| 2 | 30 | | sodium | NaCl | polysorbate |
| 3 | 30 | | phosphate | | 80 |
| 4 | 50 | | | | |
| 5 | 10 | | | 9.5% | |
| 6 | 30 | | | trehalose | |
| 7 | 50 | | | | |
| 8 | 10 | | | 150 mM | |
| 9 | 30 | | | arginine | |
| 10 | 30 | | | | |
| 11 | 30 | | | | |
| 12 | 50 | | | | |
| 13 | 10 | | | 5.0% sorbitol | |
| 14 | 30 | | | | |
| 15 | 50 | | | | |
| 16 | 10 | | | 8.5% sucrose | |
| 17 | 30 | | | | |
| 18 | 50 | | | | |
| 19 | 10 | 7.0 | 10 mM | 150 mM | 0.022% |
| 20 | | | sodium | NaCl | polysorbate |
| 21 | | | phosphate | | 80 |

Experimental stress conditions set up to observe feasibility for high concentration of anti-C5 antibody were thermal stress (25° C. for 0, 1, 2, or 4 weeks) and freeze/thaw (−70° C. for 1, 3, and 5 cycles). After being exposed to each stress condition, samples were analyzed for pH, protein concentration (UV), high molecular weight (HMW) content (SE-HPLC), and acidic content (AEX-HPLC).

TABLE 11
ΔHMW % Summary of Samples at 0, 1, 2 and 4 Weeks

| ΔHMW % | | | | | | |
|---|---|---|---|---|---|---|
| Sample | | Antibody | 25° C., Thermal Stress Condition | | | |
| No. | Stabilizer | concentration | 0 wk | 1 wk | 2 wk | 4 wk |
| 4 | NaCl | 50 mg/ml | 0.00 | 0.92 | 1.55 | 1.70 |
| 7 | Trehalose | 50 mg/ml | 0.00 | 0.86 | 1.30 | 1.62 |
| 12 | Arginine | 50 mg/ml | 0.00 | 0.01 | 0.21 | 0.35 |
| 15 | Sorbitol | 50 mg/ml | 0.00 | 0.92 | 1.40 | 1.71 |
| 18 | Sucrose | 50 mg/ml | 0.00 | 0.79 | 1.24 | 1.57 |
| 19 | NaCl | 10 mg/ml | 0.00 | 0.11 | 0.35 | 0.54 |
| 20 | NaCl | 10 mg/ml | 0.00 | 0.11 | 0.38 | 0.63 |
| 21 | NaCl | 10 mg/ml | 0.00 | 0.13 | 0.39 | 0.56 |

Under the thermal stress conditions (25° C.), the observed difference in the percentage of HMW among the formulations was directly influenced by the concentration of anti-C5 antibody (i.e., eculizumab) and the type of stabilizer. Although changes in percentage of HMW (HMW %) increased with the concentration of the anti-C5 antibody, HMW % of all arginine-containing formulations were less than that of conventional eculizumab formulation (i.e. SOLIRIS™ eculizumab formulation) (Sample Nos. 19-21). In particular, at 50 mg/ml of anti-C5 antibody, arginine-containing formulations showed great stabilizing capacity with an increase in HMW % of only 0.35%. In contrast, the average increase in HMW % in conventional eculizumab formulation (i.e. SOLIRIS™ eculizumab formulation) (Sample Nos. 19-21) was 0.58%. The increase in the percent HMW aggregate products in the stable aqueous composition comprising a 50 mg/ml of anti-C5 antibody can be reduced by about 30% or more or by about 30% to about 100% (e.g., about 30%, about 39%, about 40%, about 50%, about 60%, about 70%, about 80%, about 90%, about 95%, about 99%, about 100%, or a range between any of these values) as compared to a composition containing 10 mg/ml of the antibody formulated in 10 mM phosphate buffer, 150 mM sodium chloride, 0.022% polysorbate 80, pH 7.0 (i.e., SOLIRIS™ eculizumab formulation) as determined by SEC after four weeks of thermal stress at 25° C.

HMW % of 50 mg/ml antibody formulations containing trehalose, arginine or sucrose also were less than HMW % of the 50 mg/ml antibody formulation containing NaCl. At 50 mg/ml of anti-C5 antibody, the arginine-containing formulation showed great stabilizing capacity with an increase in HMW % of only 0.35%. In contrast, the increase in HMW % in the NaCl-containing formulation was 1.70%. Thus, the increase in the percent HMW aggregate products in the stable aqueous composition comprising a 50 mg/ml of anti-C5 antibody can be reduced by about 3% or more (e.g., about 3% to about 90%) as compared to a composition containing 50 mg/ml of the antibody in a NaCl-containing formulation as determined by SEC after four weeks of thermal stress at 25° C.

TABLE 12
ΔHMW % Summary of Samples after 0, 1, 3 and 5 F/T Cycles

| ΔHMW % | | | | | | |
|---|---|---|---|---|---|---|
| Sample | | Antibody | Freeze/Thaw Cycling | | | |
| No. | Stabilizer | Concentration | 0 | 1 | 3 | 5 |
| 4 | NaCl | 50 mg/ml | 0.00 | −0.02 | 0.04 | 0.00 |
| 7 | Trehalose | 50 mg/ml | 0.00 | −0.06 | −0.03 | −0.13 |
| 12 | Arginine | 50 mg/ml | 0.00 | −0.02 | 0.00 | −0.02 |
| 15 | Sorbitol | 50 mg/ml | 0.00 | 0.00 | 0.02 | −0.02 |
| 18 | Sucrose | 50 mg/ml | 0.00 | −0.03 | −0.02 | −0.07 |
| 19 | NaCl | 10 mg/ml | 0.00 | 2.40 | 5.83 | 8.98 |
| 20 | NaCl | 10 mg/ml | 0.00 | 1.90 | 5.80 | 8.90 |
| 21 | NaCl | 10 mg/ml | 0.00 | 1.76 | 5.73 | 7.71 |

Under the freeze/thaw cycling-induced stress, trehalose, sucrose, sorbitol, and arginine successfully hindered formation of protein aggregates in 50 mg/ml antibody formulations, which is detected as the HMW portion. Even after five freeze-thaw cycles, the HMW % increase was less than 0.1% as opposed to the 8.53% average increase observed in samples prepared with conventional eculizumab formulation (i.e. SOLIRIS™ eculizumab formulation) (Sample Nos. 19-21). The increase in the percent HMW aggregate products in the stable aqueous composition comprising a 50 mg/ml of anti-C5 antibody can be reduced by about 5% or more or by about 5% to about 100% (e.g., about 5%, about 10%, about 20%, about 30%, about 40%, about 50%, about 60%, about 70%, about 80%, about 90%, about 95%, about 99%, about 100%, or a range between any of these values) as compared to a composition containing 10 mg/ml of the antibody formulated in 10 mM phosphate buffer, 150 mM sodium chloride, 0.022% polysorbate 80, pH 7.0 (i.e., SOLIRIS™ eculizumab formulation) as determined by SEC after five cycles of freeze/thaw (−70° C./RT).

TABLE 13
ΔAcidic % Summary of Samples at 0, 1, 2 and 4 Weeks

| ΔAcidic % | | | | | | |
|---|---|---|---|---|---|---|
| Sample | | Antibody | 25° C.; Thermal Stress Condition | | | |
| No. | Stabilizer | Concentration | 0 wk | 1 wk | 2 wk | 4 wk |
| 4 | NaCl | 50 mg/ml | 0.00 | −0.59 | 0.39 | 2.89 |
| 7 | Trehalose | 50 mg/ml | 0.00 | −1.68 | −0.28 | 0.38 |
| 12 | Arginine | 50 mg/ml | 0.00 | −1.01 | 0.34 | 2.50 |
| 15 | Sorbitol | 50 mg/ml | 0.00 | −1.14 | −0.59 | 0.70 |
| 18 | Sucrose | 50 mg/ml | 0.00 | −0.68 | −0.04 | 0.11 |
| 19 | NaCl | 10 mg/ml | 0.00 | 0.31 | 1.62 | 3.30 |
| 20 | NaCl | 10 mg/ml | 0.00 | 0.48 | 1.53 | 3.70 |
| 21 | NaCl | 10 mg/ml | 0.00 | 0.78 | 1.68 | 3.62 |

Similarly, under the thermal stress conditions, though statistically significant changes in acidic content percent (acidic %) depended on the type of stabilizer, the increased acidic % in all formulations was less than that of conventional eculizumab formulation (i.e. SOLIRIS™ eculizumab formulation) (Sample Nos. 19-21), which was 3.54% (average). Among formulations with 50 mg/ml antibody concentration, sucrose-containing formulation showed stabilizing capability with a change in acidic % of 0.11%, followed by trehalose, sorbitol, and arginine with 0.38%, 0.70%, and 2.50% increases, respectively. No meaningful change in acidic % dependent on protein concentration was observable. The increase in the acidic content percent in the stable aqueous composition comprising a 50 mg/ml of anti-C5 antibody can be reduced by about 20% or more or by about 20% to about 100% (e.g., about 20%, about 29%, about 30%, about 40%, about 50%, about 60%, about 70%, about 80%, about 89%, about 90%, about 95%, about 96%, about 97%, about 100%, or a range between any of these values) as compared to a composition containing 10 mg/ml of the antibody formulated in 10 mM phosphate buffer, 150 mM sodium chloride, 0.022% polysorbate 80, pH 7.0 (i.e., SOLIRIS™ eculizumab formulation) as determined by AEX-HPLC after four weeks of thermal stress at 25° C.

Acidic % of 50 mg/ml antibody formulations containing trehalose, arginine, sorbitol, and sucrose also were less than acidic % of the 50 mg/ml antibody formulation containing NaCl as determined by AEX-HPLC after four weeks of thermal stress at 25° C. As noted above, sucrose, trehalose, sorbitol, and arginine formulations showed stabilizing capability with a change in acidic % of 0.11%, 0.38%, 0.70%, and 2.50% increases, respectively, and the NaCl formulation showed a change in acidic % of 2.89%. The increase in the acidic content percent in the stable aqueous composition comprising a 50 mg/ml of anti-C5 antibody and sucrose, trehalose, sorbitol, or arginine can be reduced by about 10% or more (e.g., about 10% to about 100%) as compared to a composition containing 50 mg/ml of the antibody formulated in NaCl as determined by AEX-HPLC after four weeks of thermal stress at 25° C.

In summary, formulation samples containing trehalose, arginine, sorbitol, or sucrose showed no significant change in HMW % and acidic % under thermal stress and freeze-thaw cycling stress conditions. Therefore, it is feasible to maintain the stability of formulations with a higher concentration with an appropriate stabilizer.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A stable aqueous composition comprising:
(a) about 10 to about 100 mg/ml of an anti-C5 antibody,
(b) a surfactant,
(c) a stabilizer, and
(d) a buffer having a pH of about 5.0 to about 7.8,
wherein the stabilizer is trehalose, sucrose, sorbitol, arginine, or a combination thereof, and
wherein the composition does not comprise at least one selected from the group consisting of dextrose, glycerin, mannitol, potassium chloride and sodium chloride.

2-3. (canceled)

4. The stable aqueous composition of claim 1, wherein the antibody comprises a light chain comprising the amino acid sequence of SEQ ID NO: 1, and a heavy chain comprising the amino acid sequence of SEQ ID NO: 2.

5. The stable aqueous composition of claim 1, wherein the antibody is eculizumab.

6-7. (canceled)

8. The stable aqueous composition of claim 1, wherein the surfactant is about 0.01% to about 0.1% (w/v) surfactant, and
wherein the surfactant is polysorbate 20 or polysorbate 80.

9-12. (canceled)

13. The stable aqueous composition of claim 1, wherein the buffer is about 1 mM to about 30 mM buffer, and
wherein the buffer comprises phosphate, histidine, or a combination thereof.

14.-26. (canceled)

27. The stable aqueous composition of claim 1, wherein the antibody neutralizes human C5 activity with an IC₅₀ of 2-4 μg/ml based on an in vitro hemolysis assay.

28-45. (canceled)

46. A stable aqueous composition consisting essentially of:
(a) about 10 mg/ml of an anti-C5 antibody,
(b) about 0.01% to about 0.1% (w/v) of a surfactant,
(c) about 1 to about 30 mM buffer having a pH of 5.5 to 7.5, and
(d) a stabilizer selected from the group consisting of trehalose, sucrose, sorbitol, arginine, and a combination thereof,
wherein the composition does not comprise i) and/or ii):
i) at least one selected from the group consisting of dextrose, glycerin, mannitol, potassium chloride and sodium chloride, and
ii) glycine.

47-55. (canceled)

56. The stable aqueous composition of claim 46, wherein the antibody is eculizumab.

57-83. (canceled)

84. A method of preparing a composition suitable for administration comprising diluting the stable aqueous composition of claim 1 with an anti-C5 antibody, to be about 10 mg/ml of the antibody when the concentration of antibody is not about 10 mg/mL, in which the stable aqueous composition comprises:
(a) about 0.01% to about 0.1% (w/v) of surfactant selected from polysorbate 20 or polysorbate 80,
(b) about 1 to about 30 mM buffer having selected from phosphate, histidine, or a combination thereof, and
(c) a stabilizer selected from the group consisting of trehalose, sucrose, sorbitol, and arginine,
wherein the concentration of the stabilizer is about 1% to about 20% (w/v) when the stabilizer is at least one selected from trehalose, sucrose, and sorbitol,
wherein the concentration of the stabilizer is about 50 mM to about 300 mM when the stabilizer is arginine, and
wherein the composition does not comprise i) and/or ii):
i) at least one selected from the group consisting of dextrose, glycerin, mannitol, potassium chloride and sodium chloride, and
ii) glycine.

85. (canceled)

86. The stable aqueous composition of claim 1, wherein the composition does not comprise glycine.

87. The stable aqueous composition of claim 1, wherein the composition contains about 10 mg/mL of the anti-C5 antibody.

88. The stable aqueous composition of claim 1, wherein the composition contains about 50 mg/ml of the anti-C5 antibody.

89. The stable aqueous composition of claim 27,
wherein the in vitro hemolysis assay comprises:
(a) diluting the composition,
(b) adding human serum to the diluted composition and incubating with a sample containing red blood cells, and
(c) measuring hemolysis.

90. The stable aqueous composition of claim 46, wherein the surfactant is selected from polysorbate 20 or polysorbate 80,
wherein the buffer is selected from phosphate, histidine, or a combination thereof,
wherein the concentration of the stabilizer is about 1% to about 20% (w/v) when the stabilizer is at least one selected from the group consisting of trehalose, sucrose, and sorbitol, and
wherein the concentration of the stabilizer is about 50 mM to about 300 mM when the stabilizer is arginine.

91. A stable aqueous composition consisting essentially of:
(a) about 30 mg/ml of an anti-C5 antibody,
(b) about 0.01% to about 0.1% (w/v) of polysorbate 80,
(c) about 1 mM to about 30 mM buffer having a pH of 5.5 to 7.5, and
(d) a stabilizer selected from the group consisting of trehalose, sucrose, sorbitol, arginine, and a combination thereof,
wherein the composition does not comprise i) and/or ii):
i) at least one selected from the group consisting of dextrose, glycerin, mannitol, potassium chloride and sodium chloride, and
ii) glycine.

92. The stable aqueous composition of claim 91, wherein the surfactant is selected from polysorbate 20 or polysorbate 80,
wherein the buffer is selected from phosphate, histidine, or a combination thereof,
wherein the concentration of the stabilizer is about 1% to about 20% (w/v) when the stabilizer is at least one selected from the group consisting of trehalose, sucrose, and sorbitol, and
wherein the concentration of the stabilizer is about 50 mM to about 300 mM when the stabilizer is arginine.

93. A stable aqueous composition consisting essentially of:
(a) about 50 mg/ml of an anti-C5 antibody,
(b) about 0.01% to about 0.1% (w/v) of a surfactant,
(c) about 1 mM to about 30 mM buffer having a pH of 5.5 to 7.5, and
(d) a stabilizer selected from the group consisting of trehalose, sucrose, sorbitol, arginine, and a combination thereof,
wherein the composition does not comprise i) and/or ii):
i) at least one selected from the group consisting of dextrose, glycerin, mannitol, potassium chloride and sodium chloride, and
ii) glycine.

94. The stable aqueous composition of claim 93, wherein the surfactant is selected from polysorbate 20 or polysorbate 80,
wherein the buffer is selected from phosphate, histidine, or a combination thereof,
wherein the concentration of the stabilizer is about 1% to about 20% (w/v) when the stabilizer is at least one selected from the group consisting of trehalose, sucrose, and sorbitol, and
wherein the concentration of the stabilizer is about 50 mM to about 300 mM when the stabilizer is arginine.


SEQUENCE LISTING (10 sequences)

SEQ ID NO: 1 - protein, 214 aa, Artificial Sequence [sequence omitted]

SEQ ID NO: 2 - protein, 448 aa, Artificial Sequence
  anti-C5 antibldy heavy chain
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 109 aa, Artificial Sequence
  anti-C5 antibldy light chain variable region
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRT

SEQ ID NO: 4 - protein, 123 aa, Artificial Sequence
  anti-C5 antibldy heavy chain variable region
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSA

SEQ ID NO: 5 - protein, 11 aa, Artificial Sequence
  CDRL1
GASENIYGALN

SEQ ID NO: 6 - protein, 7 aa, Artificial Sequence
  CDRL2
GATNLAD

SEQ ID NO: 7 - protein, 9 aa, Artificial Sequence
  CDRL3
QNVLNTPLT

SEQ ID NO: 8 - protein, 5 aa, Artificial Sequence
  CDRH1
NYWIQ

SEQ ID NO: 9 - protein, 17 aa, Artificial Sequence
  CDRH2
EILPGSGSTEYTENFKD

SEQ ID NO: 10 - protein, 13 aa, Artificial Sequence
  CDRH3
YFFGSSPNWYFDV